(19) United States
(12) Patent Application Publication
(10) Pub. No.: US 2023/0127232 A1
(43) Pub. Date: Apr. 27, 2023

(54) FLOW ASSAY CARTRIDGE

(71) Applicant: Kenota Inc., Kitchener (CA)

(72) Inventors: Rob Rennie, Stratford (CA); Christopher J. Harder, Osborne Corner (CA); Yiheng Qin, Mississauga (CA); Kien Vu, Nepean (CA); Bill Dicke, Aylmer (CA); Calvin James Brereton, Kitchener (CA)

(21) Appl. No.: 17/906,924

(22) PCT Filed: Apr. 2, 2021
(86) PCT No.: PCT/CA2021/050451

§ 371 Date: Sep. 21, 2022

Related U.S. Application Data
(60) Provisional application No. 63/004,670, filed on Apr. 3, 2020.

(51) Int. Cl. B01L 3/00

(52) U.S. Cl. CPC B01L 3/502715; B01L 2200/025; B01L 2200/028; B01L 2300/043; B01L 2300/0825; B01L 2300/0851; B01L 2300/042; B01L 2200/10; B01L 2400/0406

(57) ABSTRACT

A flow assay cartridge for housing and protecting a flow assay membrane or lateral flow test strip which can be vertically stacked and is adapted for high-throughput automated lateral flow assay testing and analysis. The flow assay cartridge comprises a base and lid for receiving the flow assay membrane, and top and bottom engagement features such that two or more flow assay cartridges can be releasably adjoined in a vertical orientation such that they can be easily handled by an automated assay apparatus.

### CROSS-REFERENCE TO RELATED APPLICATIONS

This application claims priority to U.S. provisional patent application US63/004,670 filed 3 Apr. 2020, the contents of which are hereby incorporated by reference herein in its entirety.

### FIELD OF THE INVENTION

The present invention pertains to a flow assay cartridge for housing and protecting a flow assay membrane or lateral flow test strip which can be vertically stacked and is adapted for high-throughput automated lateral flow assay testing and analysis. The flow assay cartridge comprises top and bottom engagement features such that two or more flow assay cartridges can be releasably adjoined such that they can be vertically stacked and easily handled by an automated assay apparatus.

### BACKGROUND

Immunological flow assay tests, also referred to as immunoassays, exist for a wide array of target analytes including monitoring ovulation, detecting infectious disease organisms, analyzing drugs of abuse, and measuring other analytes important to human physiology such as the presence of microorganisms, pharmaceuticals, hormones, viruses, antibodies, nucleic acids, and other proteins. In serum assays, antibodies can be detected on flow assay membranes as indicators of various disease states and immunological status by detecting the formation of a complex between a detector particle that is free in the sample stream and a capture reagent that is bound to the membrane at a test line. Flow assay devices have also been employed for qualitative, semi-quantitative, and quantitative measurement of small amounts of materials in biological samples in healthcare, veterinary testing, agricultural applications, food safety, environmental testing, and product quality evaluation. In point-of-care diagnostics, some examples of samples which may be useful for testing are blood, milk, urine, serum, plant materials or extracts, and food samples.

While the first flow assay tests presented qualitative results based on the presence or absence of a signal line, test design has progressed toward semi-quantitative and quantitative assays, and flow assay membranes are now being used with the integration of hand-held readers and high throughput analyzers and laboratory or point-of-care devices. Various types of analyzers can enable concurrent diagnostic testing at a plurality of flow assay devices and provide an integrated and robust sample-processing system with concurrent testing such that a plurality of flow assay devices can be incubated and processed concurrently. Flow assay membranes and associated cartridges can be designed that use small test volumes such that results can be obtained using high performance visualization to provide qualitative and quantitative results. A flow assay cartridge houses and protects a flow assay membrane, also known as a lateral flow test strip, before, during, and after flow assay analysis, and is particularly useful in combination with high throughput analyzers.

Automated systems that can process a multitude of lateral flow assay devices at a time can decrease sample turn-around time and provide high throughput in assay testing and analysis. One example lateral flow assay apparatus for use in clinical diagnostics is described in U.S. Pat. No. 9,709,562 to Jakubowicz et al. in which a plurality of lateral flow assay devices can be retained in an automated assay analyzer. For automated systems which can process multiple flow assays cartridges at a time with test automation, robust flow assay cartridges which can be easily handled provide reliability, safety, and reproducibility in a high throughput system. Such automated systems can be deployed as point of care diagnostic systems for use by technicians while still providing reliable and reproducible results.

There remains a need for a flow assay cartridge for an automated lateral flow assay testing and analysis.

This background information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present invention is to provide a flow assay cartridge for housing and protecting a flow assay membrane or lateral flow test strip which is adapted for high-throughput automated lateral flow assay testing and analysis.

In an aspect there is provided a flow assay cartridge comprising: a cartridge base; a cartridge lid engageable with the cartridge base; a bottom engagement feature on the cartridge base; and a top engagement feature, wherein the bottom engagement feature of a first flow assay cartridge can be engaged with the top engagement feature of a second flow assay cartridge positioned below the first flow assay cartridge for releasable sliding engagement of the second flow assay cartridge to the first flow assay cartridge.

In another aspect there is provided a flow assay cartridge comprising: a cartridge base; a cartridge lid engageable with the cartridge base; a bottom engagement feature; and a top engagement feature for releasable engagement with the bottom engagement feature of a second flow assay cartridge positioned above the flow assay cartridge.

In an embodiment of the cartridge, the releasable engagement of the top engagement feature with the bottom engagement feature of the second flow assay cartridge is a sliding engagement.

In another embodiment of the cartridge, the top engagement feature and bottom engagement feature comprise a rail track and a complementary rail guide.

In another embodiment of the cartridge, the top engagement feature comprises a rail track and the bottom engagement feature comprises at least one rail guide.

In another embodiment of the cartridge, the bottom engagement feature and the top engagement feature are friction-fit engagement features, snap-fit engagement features, or a combination thereof.

In another embodiment of the cartridge, the cartridge is releasably vertically stackable with a plurality of similar cartridges.

In another embodiment of the cartridge, wherein when the flow assay cartridge is vertically stacked with the second flow assay cartridge, the cartridge lid is covered by the cartridge base of the second flow assay cartridge.

In another embodiment, the cartridge further comprises a flow assay membrane in the cartridge.

In another embodiment, the cartridge further comprises features for engagement with an analyzer.

In another embodiment of the cartridge, the cartridge lid is reversibly engaged to the cartridge base.

In another embodiment of the cartridge, the cartridge lid has a plurality of apertures.

In another aspect there is provided a method of flow assay automation comprising: disengaging a first assay cartridge from a vertically engaged stack of assay cartridges; applying sample to the first assay cartridge to begin the assay; and reengaging the first assay cartridge to another assay cartridge in the plurality of vertically engaged stack of assay cartridges.

In an embodiment, disengaging of a first assay cartridge from the vertically engaged stack of assay cartridges is done by an automated device.

In another embodiment, disengaging of a first assay cartridge from the vertically engaged stack of assay cartridges is done by sliding the first assay cartridge away from the stack of assay cartridges.

In another embodiment the method further comprises analysing the results of the assay.

In another aspect there is provided a diagnostic test device comprising: a flow assay membrane; and a flow assay cartridge for receiving the flow assay membrane, the cartridge comprising: a cartridge base; a cartridge lid engageable with the cartridge base; a bottom engagement feature; and a top engagement feature for releasable engagement with the bottom engagement feature of a second flow assay cartridge positioned above the flow assay cartridge.

In another aspect there is provided a diagnostic test device comprising: a flow assay membrane; and a flow assay cartridge for receiving the flow assay membrane, the cartridge comprising: a cartridge base; a cartridge lid engageable with the cartridge base; a bottom engagement feature on the cartridge base; and a top engagement feature, wherein the bottom engagement feature of a first flow assay cartridge can be engaged with the top engagement feature of a second flow assay cartridge positioned below the first flow assay cartridge for releasable sliding engagement of the second flow assay cartridge to the first flow assay cartridge.

In an embodiment of the test device, the releasable engagement is a sliding engagement.

In another embodiment, the test device further comprises a mounting locus where an analyzer component can engage with the assay cartridge for secure transport in the analyzer.

In another embodiment, the bottom engagement feature and the top engagement feature are friction-fit engagement features, snap-fit engagement features, or a combination thereof.

In another embodiment, the bottom engagement feature and the top engagement feature comprise at least one rail track and at least one rail guide.

In another embodiment, the bottom engagement feature and the top engagement feature comprise at least two rail tracks and at least two rail guides.

### BRIEF DESCRIPTION OF THE FIGURES

For a better understanding of the present invention, as well as other aspects and further features thereof, reference is made to the following description which is to be used in conjunction with the accompanying drawings, where:

FIG. 1 is an isometric view of multiple engaged flow assay cartridges;

FIG. 2 is an isometric view of the bottom of a flow assay cartridge;

FIG. 3 is a top isometric view of a lid and base of a flow assay cartridge;

FIG. 4 is an isometric cross-sectional view of a flow assay cartridge;

FIG. 5 is a bottom isometric view of a lid of a flow assay cartridge;

FIG. 6 is a top isometric view of the base of a flow assay cartridge;

FIG. 7a is an isometric view of the closing side of a flow assay cartridge;

FIG. 7b is an isometric view of the opening side of a flow assay cartridge;

FIG. 8 is a side cross-sectional view of a flow assay cartridge;

FIG. 9 is an isometric view of a flow assay membrane;

FIG. 10 is a front view of a vertically engaged stack of flow assay cartridges; and

FIG. 11 is a front view of a cartridge base with engagement features.

### DETAILED DESCRIPTION OF THE INVENTION

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

The term “comprising” as used herein will be understood to mean that the list following is non-exhaustive and may or may not include any other additional suitable items, for example one or more further feature(s), component(s) and/or element(s) as appropriate.

As used herein, the terms “connect” and “connected” refer to any direct or indirect physical association between elements or features of the present disclosure. Accordingly, these terms may be understood to denote elements or features that are partly or completely contained within one another, attached, coupled to, disposed on, joined together, in communication with, operatively associated with, or fluidically coupled to, etc., even if there are other elements or features intervening between the elements or features described as being connected.

The terms “flow assay membrane” and “lateral flow test strip” are used interchangeably herein to refer to a generally planar substrate used to elute a component of interest. The flow assay membrane is preferably made from one or more suitable porous or non-porous materials with surface properties that support capillary like flow. The flow assay membrane receives a sample and/or sample or buffer fluid and includes a fluid transport or flow path along which various areas or sites are provided for supporting one or more reagents, filters, and the like and through which sample traverses under the influence of capillary action or other forces. Flow assay membranes can include but are not limited to thin-film or “dry slide” test elements, lateral flow assay devices, microfluidic paper-based analytical devices (μPADs), vertical flow assay devices, and chromatography devices. The flow assay membrane also refers to the carrier or matrix or combination of membranes to which a sample is added, and on or in which the determination is performed, or where the reaction between analyte and reagent takes place. Membranes come in a wide variety of sizes, with more common sizes of 2-10 mm wide and from 30-100 mm long.

The term “sample” as used herein, refers to a volume of a liquid, fluid, solution, or suspension, intended to be subjected to qualitative or quantitative determination of any of its properties or components, such as the presence or absence of a component, the concentration of a component, etc. Typical samples in the context of the present invention as described herein are derived from human or animal bodily fluids such as but not limited to blood, plasma, serum, lymph, urine, saliva, semen, amniotic fluid, gastric fluid, phlegm, sputum, mucus, tears, stool, etc. Other types of samples are derived from human or animal tissue samples where the tissue sample has been processed into a liquid, solution, or suspension to reveal particular tissue components for examination. Other non-limiting examples of samples that can be used are environmental samples, food industry samples, and agricultural samples.

The terms “analyte,” “analyte of interest,” and “species of interest” in this disclosure refer to any and all clinically, diagnostically, or relevant chemical or biological analytes present in a sample. Analytes of interest can include, but are not limited to antibodies, hormones, molecules, antigens, organic chemicals, biochemicals, and proteins. Some non-limiting examples of antibodies include antibodies that bind food antigens, and antibodies that bind infectious agents such as virus and bacteria, for example anti-CCP, anti-streptolysin-O, anti-HIV, anti-hepatitis (anti-HBc, anti-HBs etc), antibodies against Borrelia, and specific antibodies against microbial proteins.

The term “analyzer” as used herein, refers to any apparatus enabling the automated processing of various analytical test or flow assay devices, and in which a plurality of test devices can be processed. The analyzer can comprise a plurality of components configured for loading, incubating, testing, transporting, and evaluating a plurality of analytical test elements in an automated or semi-automated fashion and in which sample and/or other fluids may be automatically dispensed and processed substantially without user intervention. Analyzers include but are not limited to clinical diagnostic apparatus and point-of-care type devices.

The term “reaction” as used herein, refers to any interaction which takes place between components of a sample and at least one reagent or reagents on or in, or added to, the substrate of the test device, or between two or more components present in the sample. The term “reaction” is used to define the reaction taking place between an analyte and a reagent on the test device as part of the qualitative or quantitative determination of the analyte.

Herein is described flow assay cartridge for housing and protecting a flow assay membrane or lateral flow test strip which is adapted for high-throughput automated lateral flow assay testing and analysis. The flow assay cartridge comprises engagement features such that two or more flow assay cartridges can be releasably adjoined in a vertical stack or configuration such that multiple flow assay cartridges can be easily handled by an automated assay apparatus. The presently described assay cartridge can be used in automated or semi-automated lateral flow assay analyzers and point-of-care diagnostic devices. The flow assay cartridge houses and protects a flow assay strip or flow assay membrane and provides robustness during manufacture, distribution, storage, and for transport and movement of the flow assay strip in an automated analyzer. A solid assay cartridge is further capable of being moved by one or more moving mechanisms in an analyzer without damage, disruption, or contamination to the planar substrate of the flow assay membrane strip and detection area within.

The following exemplary embodiments relate to the configuration and design of a flow assay cartridge which is vertically connectable to one or more other flow assay cartridges.

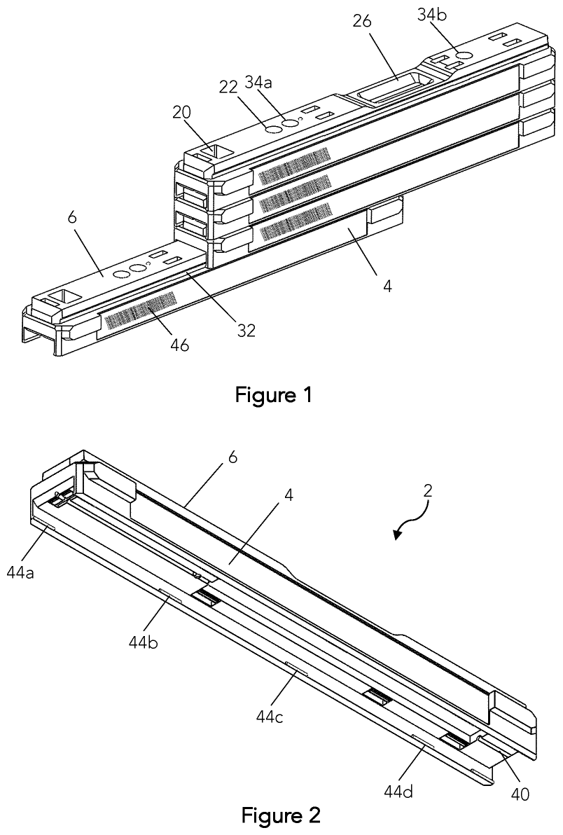

FIG. 1 is an isometric view of a stack of four flow assay cartridges with slidable vertical engagement with the bottom flow assay cartridge in the stack slide out relative to the cartridges above it. Each individual assay cartridge has a cartridge base 4 or base which houses a flow assay membrane and a cartridge lid 6. The cartridge base 4 is capable of receiving and supporting a flow assay membrane for diagnostic flow assay testing and can have a variety of interior configurations for supporting, holding, and maintaining the integrity of the flow assay membrane. The cartridge lid 6 comprises a plurality of apertures that can be used for receiving various samples, reagents and/or fluids, and for visualising the flow assay membrane at various times including before, during, and after the flow assay membrane has been eluted. The exemplified cartridge comprises a buffer port 20, a sample addition port 22, two quality control (qc) windows 34a and 34b, and a results window 26 through which to view the results of the assay. It is understood that the number and placement and design of apertures in the cartridge lid 6 can be varied depending on the assay design desired and the structure of the flow assay membrane.

The flow assay cartridge comprises vertical engagement features to enable vertical engagement of two or more cartridges such that cartridges can be engaged and disengaged from one another. Without being bound by theory, it is has been found that vertical stacking of engaged flow assay cartridges can enable efficient cartridge storage, packing, orientation, and handling, enabling movement and transport of a stack of assay cartridges as a single unit. In a laboratory or point-of-care setting, handling of multiple assay cartridges at a time saves time in handling and reduces the risk of error. Cartridges can also be packaged together and supplied in an engaged vertical stack for ease of handling. In addition, protection of open apertures on the lid of each assay cartridge during transport and incubation is provided by the cartridge above in the vertical stack. By covering the apertures on the cartridge lid with the cartridge base of the assay cartridge above it, the flow assay membrane inside the flow assay cartridge can be provided with protection during transport, including in an automated or semi-automated analyzer and/or during handling, as well as locally controlled humidity during assay running. In particular, covering the apertures on the top of the flow assay cartridge during elution can delay evaporation of the buffer or eluent and also provide a local chamber which is protected from contamination and/or humidity loss during elution of the assay test.

The flow assay cartridge shown has a sliding rail track 32 on the cartridge lid 6 which serves as a top engagement feature, and is configured to receive and engage with one or bottom engagement features (not shown) on the bottom of the assay cartridge above it, where the bottom engagement features slidingly fit inside the rail track 32. In the shown embodiment rail track 32 on a bottom assay cartridge engages in a sliding arrangement with a rail guide (not shown) on the base of the cartridge above it such that the rail guide slides into the rail track 32, forming a secure but reversible sliding engagement. The rail track and engaged rail guide design can enable linear sliding of the engaged flow assay cartridges in both directions, in particular enabling cartridge sliding and/or separation from both the buffer port 20 end of the cartridge and the opposite end of the flow assay cartridge. Optionally one or more stopping feature can be provided either on the cartridge lid 6 or cartridge base 4 to restrict sliding of the slide rail 32 relative to the rail guide in one direction only, or prevent complete separation of the vertically stacked assay cartridges as desired. Any combination of top engagement feature and bottom engagement feature that provides sliding releasable engagement of two vertically stacked assay cartridges can be employed, including various configurations of linear sliding engagement features as shown, as well as friction fit engagement features, and combinations thereof. Rail track 32 shown is a dovetail slide, with matching dovetail rail guides on the cartridge base. A variety of other joins are conceivable, including but not limited to a ball rail and track with circular cross-section, and a continuous or semi-continuous rail guide with two more protrusions to provide loci for slidingly mating with the rail track. It is understood that the top engagement feature and bottom engagement feature can be anywhere on the cartridge which enable vertical stacking of two cartridges in a reversibly securable manner. In a linear motion sliding fit the two features of the longitudinal rail track (elongated channel) and a complementary rail guide (feature that can be slidingly received in the channel) can each be either on the top or bottom of the cartridge, providing that together they can be joined in a releasable but securable way. Friction fit engagement of two cartridges can also be used, including using malleable or deformable materials for the slide and/or rail such that two cartridges can be reversibly disengaged and re-engaged and aligned using a snapping motion, or features which provide the same functionality, such as, for example, two or more deformable post and/or aperture arrangements. The assay cartridge can also have one or more optional barcode 46, which can be any digital data stored as an image that can be read by an optical reader. Alternatively, the assay cartridge can have one or more other identification tags such as, for example, an RFID tag or electromagnetic label.

FIG. 2 is an isometric view of the bottom of a flow assay cartridge 2 having an engaged cartridge lid 6 and cartridge base 4. The bottom of the assay cartridge has slidable longitudinal bottom engagement features comprising a plurality of rail guides 44a, 44b, 44c, 44d that fit slidably into the rail track in the top of the assay cartridge. The cartridge base can also optionally have one or more mounting locus 40 to provide a location where an analyzer component can engage with the assay cartridge for secure transport in the analyzer. The mounting locus can be a sliding or friction fit engagement location for mounting with a complementary feature on the analyzer and can be, for example, a linear slide, track, or guide, or one or more aperture for receiving a complementary peg or protrusion in the analyzer. These engagement features can be included as part of the base or lid interchangeably. Complementary protrusions in a movement mechanism of an automated analyzer can engage with the mounting locus 40 to provide a site for secure reversible engagement for movement of the cartridge in an automated or semi-automated analyzer. It is understood that any configuration of mounting locus or other mounting features on the cartridge would have utility in reversible coupling of the assay cartridge with the analyzer movement mechanism such that the assay cartridge can be separated from another assay cartridge with which it is engaged, moved by the movement mechanism in the analyzer for application of sample, mobile fluid, or any other fluid, for visualization and analysis of the assay results, and for generally moving the assay cartridge around the analyzer. When the assay cartridges have a vertical stacking arrangement as shown in FIG. 1, a movement mechanism of an automated analyzer can engage with the analyzer engagement apertures complementary to the mounting locus 40, and slidably disengage the assay cartridge from the assay cartridge above it. Furthermore any configuration of mounting features on the cartridge can fit together with a specific physical interference such that sliding friction keeps vertical cartridge stacks substantially together during handling outside of the automated analyzer. In particular, the engagement of two assay cartridges along the bottom engagement feature of a first cartridge and a top engagement feature of a second cartridge can have sufficient friction to maintain the cartridges in a vertically stacked configuration but still allow sliding movement when sufficient force is applied.

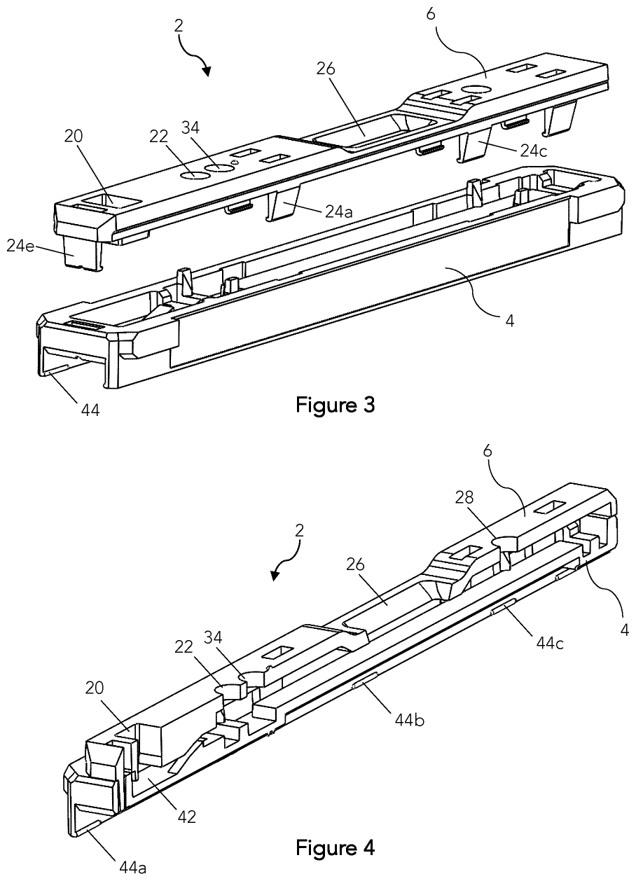

FIG. 3 is a top isometric view of a cartridge lid 6 and cartridge base 4 of a two-piece flow assay cartridge 2. The cartridge base 4 has a base, two long sides, and two short sides, and supports a flow assay membrane which is held in place in a cavity above the cartridge base bottom, and can be further held in place with engagement of the cartridge base with the cartridge lid 6. The flow assay cartridge 2 encapsulates a lateral flow assay membrane or strip while enabling addition of buffer, sample, any additional agents, and detection of reaction, while protecting the flow assay membrane during cartridge handling. Separate cartridge lid 6 engages with cartridge base 4 to secure and protect the lateral flow assay membrane inside the cartridge. In one embodiment the cartridge lid 6 can be secured to the cartridge base 4 using a combination of clasps and clasp apertures, as well as friction due to interference of engagement features to prevent cartridges freely sliding apart. As shown, clasps 24a, 24c, 24e (and other clasps on the opposite side of the cartridge lid) in the cartridge lid 6 engage with complementary clasp apertures in the cartridge base 4 to provide secure sites of engagement of the cartridge lid 6 with the cartridge base 4. Other clasping and complementary clasping arrangements are also possible, including post and aperture clasps, clips, and other friction-fit clasps, optionally with locking features. The cartridge lid 6 can also be reversibly or irreversibly engaged with the cartridge base 4, and selection and design of the complementary clasp features on the cartridge base and lid will depend on the desired setup of the assay. A hinge or permanent attachment feature of the lid and cartridge can also be used to align and engage the cartridge lid and cartridge base.

A results port 26 in the cartridge lid 6 is positioned around or above the detection area to enable one or more detector to detect reaction in the detection area of the flow assay membrane inside the cartridge. Various configurations of lateral flow assay devices are known, including but not limited to variation in device dimensions, materials, porosity of the substrate, presence or absence of topographical features on the substrate, channel shape and configuration, and method of manufacturing of the channel and/or flow assay membrane. The cartridge lid 6 also provides various ports for addition of mobile fluid, sample, reagent, binding agents, detection agents, control binding partners, labeled antibodies, and other materials for running the desired assay, and for detection of presence or absence of a component. Buffer port 20 can be used for addition of mobile fluid inside the cartridge and into a buffer well and/or directly onto the flow assay membrane. One or more sample addition port 22 is used for adding one or more samples and/or reagents through the cartridge lid to the flow assay membrane. The cartridge lid can also have one or more control or quality control windows 34 or apertures to enable visualization of the flow assay membrane inside the cartridge to confirm the validity of an assay test, to confirm the presence or absence of a particular substance or structure, or to confirm the integrity of the flow assay membrane before, during, or after the assay is run.

In the shown two piece flow assay cartridge, a rail track is formed adjacent the mating surface between the cartridge lid 6 and the cartridge base 4 upon engagement. Rail guide 44 in the bottom of the cartridge base 4 serves as a bottom engagement feature and fits into the rail track formed between the engaged cartridge lid 6 and cartridge base 4. The cartridge base can comprise multiple individual or continuous guiding engagement features or guides 44, and there are preferably at least two short guides or at least one long guide on each of the long sides of the bottom of the cartridge base 4. In one embodiment the rail guides comprise a plurality of protrusions from the base of the cartridge and the rail track is configured to receive the rail guides. In another embodiment the rail guide is a continuous protrusion and the rail track has one or more indentations configured to fit and engage with the rail guide(s). Other sliding engagement mechanisms can be used, including but not limited to sliding dovetail track and guide(s), and sliding tongue and groove track and guide(s). In another alternative, engagement between two cartridges can be a releasable friction fit, and comprise complementary protrusions and apertures wherein alignment of the protrusions and apertures provide a releasable connection between two cartridges. Any configuration of engagement features can be designed such that vertically stacked cartridges stay together during handling, but are easily separated by the analyzer movement mechanism.

FIG. 4 is an isometric cross-sectional view of an engaged cartridge base 4 and cartridge lid 4 of a flow assay cartridge 2, showing the internal structure of the cartridge. The cartridge base 4 shown has rail guides 44a, 44b, 44c which allow slidable vertical engagement in a rail track of another flow assay cartridge engaged below in a vertical arrangement. Buffer port 20 provides access through the cartridge lid 6 to buffer well 42. Sample addition port 22 and quality control window 34, results window 26, and additional control window 28 provide access to the flow assay membrane housed inside cartridge for application of materials, as well as for testing and analysis purposes. Flow assay membranes which can be received in the assay cartridge and optionally supported by the floor of the cartridge base are generally narrow and long, and sample is applied at one of the end of the membrane and carried by a mobile fluid to a detection region. Various chromatographic and immunoassays are known in the art and can be used with the presently described assay cartridge.

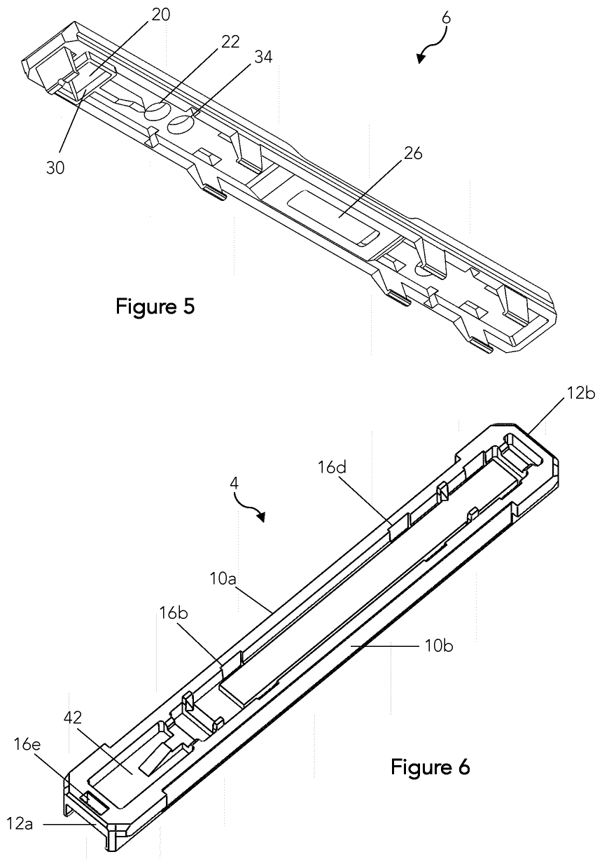

FIG. 5 is a bottom isometric view of a removable cartridge lid 6 of a flow assay cartridge. Cartridge lid 6 shown has a plurality of apertures or windows for visualization or detection of the flow assay membrane, as well as a plurality of ports for enabling physical contact with and application of materials, substances, fluids, and/or samples with the flow assay membrane. Shown are buffer port 20, sample addition port 22, quality control window 34, and results window 26, however it is understood that other cartridge lid designs may have different numbers and types of ports and apertures depending on the design and requirements of the assay. These components allow for samples and liquids to be added and results to be analysed from the cartridge. one or more optional membrane guide can assist in positioning the flow assay membrane in the right orientation and in the preferred position inside the cartridge. In particular, membrane guide 30 shown extends from the bottom side of the cartridge lid at the buffer port 20 to push down on the conjugate pad of the flow assay membrane when the cartridge lid 6 is engaged with a cartridge base to angle the conjugate pad such that it makes contact with the buffer well and any buffer inside the buffer well to facilitate fluid flow.

FIG. 6 is a top isometric view of the cartridge base 4 of a flow assay cartridge with a removable cartridge lid. The cartridge base 4 has two cartridge long sides 10a, 10b and two cartridge short sides 12a, 12b. The cartridge base 4 has a plurality clasp apertures 16a, 16b, 16c (only three of which are labeled) to facilitate the attachment of clasps in the cartridge lid 6. Buffer well 42 receives running buffer or fluid and used by the flow assay membrane for running of the assay.

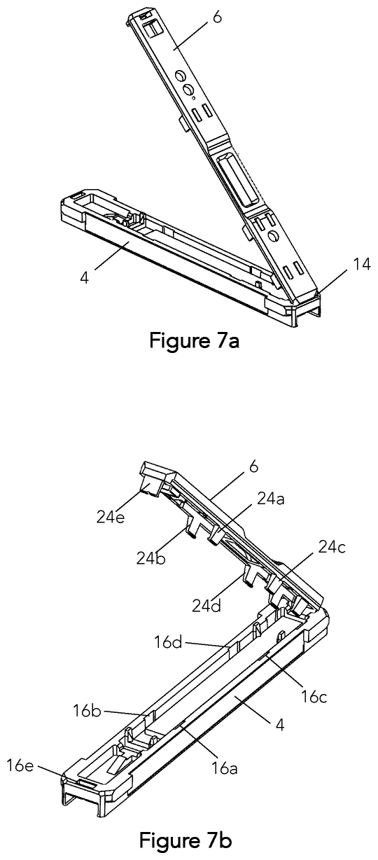

FIG. 7a is an isometric view of the closing side of a flow assay cartridge with a hinged cartridge lid. Cartridge lid 6 is aligned with cartridge base 4 through hinge 14. The hinge, working with other components, facilitates a proper alignment to firmly attach the cartridge lid 6 to the cartridge base 4.

FIG. 7b is an isometric view of the opening side of a flow assay cartridge with cartridge lid 6 and cartridge base 4 attached by a hinge. Clasps 24a, 24b, 24c, 24d, 24e align with clasp apertures 16a, 16b, 16c, 16d, 16e, respectively, to secure the cartridge lid 6 to the cartridge base 4. Although this configuration is shown with a single construction hinged flow assay cartridge it is understood that a similar configuration of clasps and complementary clasp apertures can be employed in a two-piece construction.

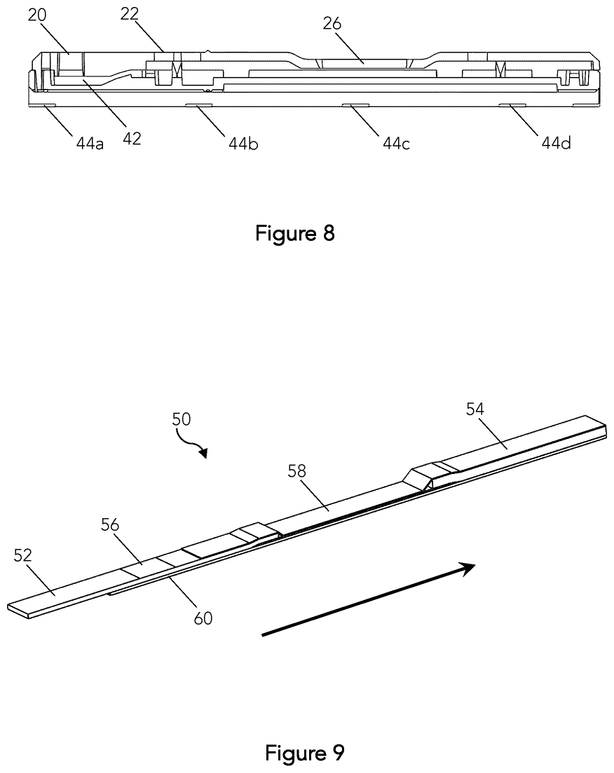

FIG. 8 is a side cross-sectional view of a flow assay cartridge for receiving a flow assay membrane. Buffer port 20 receives running fluid into buffer well 42 which stores running buffer. Sample addition port 22 is an aperture for introducing sample to the flow assay membrane. Results window 26 provides visual access to the detection area of the flow assay membrane where results can be visualized or imaged. One or more additional control window or quality control window can be present in the cartridge lid to confirm the presence of components prior to the assay or to confirm the validity of the test results once the test is finished. Rail guides 44a, 44b, 44c, 44d facilitate the vertical engagement of another assay cartridge below the one shown by engaging with a longitudinal rail track on the assay cartridge below.

FIG. 9 is an isometric view of an example of a flow assay test strip 50 or flow assay membrane that can be used with the presently described flow assay cartridge. In a lateral flow assay, the introduction of sufficient buffer or fluid sample to a conjugate pad 52 or sample addition area on a flow assay membrane spontaneously induces capillary flow along the assay membrane toward the detection area of the membrane. The direction of fluid flow along the membrane, also referred to as the fluid flow path, is shown by the arrow. The lateral flow or assay test strip is referred to in the following description in terms of the exemplary embodiment shown, however it will be readily apparent that other flow assay test strip device designs and possible variants of these designs could also be similarly configured for interrelationships with the presently described flow assay cartridge, particularly in an automated analyzer, as herein described. Test strip shown comprises, in the direction of fluid flow, a conjugate pad 52, a sample addition area 56, a detection area 58, and a wicking area 54. In an alternative design, sufficient sample and mobile fluid can be applied directly to a sample addition area or sample pad to provide adequate capillary flow in the membrane of the test strip without the requirement for additional buffer or running fluid. In the embodiment shown, conjugate pad 52 at the first end of the fluid flow path draws sample fluid in the desired direction along the lateral flow test strip from a buffer well or buffer port in the cartridge. A wick at the wicking area 54 provides a capillary force to draw up and move mobile fluid or buffer into the membrane of the test strip and through the sample addition area 56 of the test strip. The wicking area 54 can include a porous material such as, for example, nitrocellulose. Conjugate pad 52 is optionally bendable, shown extending off from an optional solid support 60, to accommodate a lowered buffer well in the assay cartridge base and further positioned by an optional wick guide in the assay cartridge base and/or lid. Obvious asymmetry in the design of the flow assay strip also provides ease of assembly of the flow assay strip within the assay cartridge and provides a directionality of the flow path so that the flow assay strip is properly aligned inside the cartridge. Optionally a hydrophilic foil or layer can be positioned directly onto at least a portion of the assay membrane to enhance the overall flow rate or process time of a sample applied to the flow assay device. The test strip can also optionally comprise one or more flow channels, optionally cut or pressed into the surface of the membrane substrate. The fluid flow path may also include additional separate areas containing one or more reagents, antibodies, or detection conjugate, as well other areas or sites along the fluid path that can be utilized used for washing of the sample and any bound or unbound components thereof. The assay membrane can also be optionally treated to adjust the sample properties, such as, for example, by pH level or viscosity. An optional lid or cover can be placed on top of the test strip downstream the sample addition area 56 as a physical protection for downstream section of the lateral flow test strip 50, with transparency to the test detection device in the detection area 58 such that the results of the assay can be detected without removing the flow assay strip from the cartridge. Detection area 58 comprises an immobilized binding species capable of binding an analyte of interest in a sample such that upon presence of the analyte of interest in an applied sample, with optional addition of a detectable species, the analyte of interest binds to the immobilized binding species and can be detected.

In use, sample addition area 56, also commonly referred to as a sample pad, receives sample, optionally via a dispenser in an automated analyzer, through a sample port in the cartridge lid. Sample applied to the sample addition area 56 is picked up by buffer drawn into the test strip 50 and flows on the substantially planar substrate of the assay membrane from the sample addition area under the capillary force created along the fluid flow path extending through the reaction area or detection area 58 on the assay membrane substrate towards the wicking area 54. One or more reagent or detection agent can either be added to or pre-loaded onto the membrane before or during the running of the assay in a location on the membrane between the sample addition area 56 and upstream the detection area 58, which in some immunoassay devices is referred to as a conjugate release area. In one example, the reagent addition area may be used to add an interrupting reagent that can be used to wash the sample and other unbound components present in the fluid flow path into wicking area 54. Reagent can either be added in the reagent area prior to use and potentially dried on the reagent area, added to the reagent area just prior to use using a reagent metering device on the analyzer, or both. The reagent can also be added via an optional reagent metering device. Reagents that can be added include but are not limited to binding partners such as antibodies or antigens for immunoassays, detection agents, conjugated antibodies, tagging molecules, fluorophores, biomarker specific antibodies, DNA and RNA aptamers with or without resonance energy transfer (RET) pairs and respective target analytes, substrates for enzyme assays, probes for molecular diagnostic assays, and auxiliary materials such as materials that stabilize the integrated reagents, materials that suppress interfering reactions, and the like. Generally, one of the reagents useful in the reaction bears a detectable signal as discussed herein. In some cases, the reagents may react with the analyte directly or through a cascade of reactions to form a detectable signal such as a colored or fluorescent molecule. In one preferred embodiment, the reagent area includes conjugate material. The term “conjugate” means any moiety bearing both a detection element and a binding partner. In use, a fluid sample is introduced to the sample addition area 56 in the device, and will flow within the fluid flow region to one or more test lines and one or more control lines on the detection area 58. The detection or test area includes one or more reagents reactive with or useful to detect a target component within the sample area. In an immunoassay in particular, as fluid moves downstream membrane conjugated antibodies are carried on the conjugate pad and the targets bind with their matching antibody. The detection area 58 comprises one or more test lines and one or more control lines and results or reaction occurs on the detection area 58 which can be detected through the results window in the assay cartridge. The test strip 50 can also comprise an optional filter material which can be placed within and/or downstream the sample addition area 56 to filter particulates from the sample, for example to filter or trap blood cells or particulate matter from blood so that added plasma can travel through the device.

Components of the flow assay devices such as the physical structure of the device described herein can be prepared from, for example, copolymers, blends, laminates, metallized foils, metallized films or metals, waxes, adhesives, or other suitable materials known to the skilled person, and combinations thereof. Alternatively, device components can be prepared from copolymers, blends, laminates, metallized foils, metallized films or metals deposited on any one or a combination of the following materials or other similar materials known to the skilled person: paraffins, polyolefins, polyesters, styrene containing polymers, polycarbonate, acrylic polymers, chlorine containing polymers, acetal homopolymers and copolymers, cellulosics and their esters, cellulose nitrate, fluorine containing polymers, polyamides, polyimides, polymethylmethacrylates, sulfur containing polymers, polyurethanes, silicon containing polymers, other polymers, glass, and ceramic materials. Alternatively, components of the device can be made with a plastic, polymer, elastomer, latex, silicon chip, or metal. In one example, the elastomer can comprise polyethylene, polypropylene, polystyrene, polyacrylates, silicon elastomers, or latex. Alternatively, components of the device can be prepared from latex, polystyrene latex or hydrophobic polymers. In one example, the hydrophobic polymer can comprise polypropylene, polyethylene, or polyester. Alternatively, components of the device can comprise TEFLON®, polystyrene, polyacrylate, or polycarbonate. Alternatively, device components can be made from plastics which are capable of being embossed, milled or injection molded or from surfaces of copper, silver and gold films upon which may be adsorbed various long chain alkanethiols. The structures of plastic which are capable of being milled or injection molded can comprise, for example, a polystyrene, a polycarbonate, a polyacrylate, or cyclo-olefin polymer.

The present cartridge system is particularly useful for immunoassay formats which are typically sandwich assays wherein the membrane is coated with a capture antibody, sample is added, and any antigen present binds to the capture antibody. In immunoassays, a detecting antibody binds to antigen in the sample, an enzyme-linked secondary antibody binds to the detecting antibody or to the antigen, and a substrate in the fluid is converted by the enzyme into a detectable form. In an automated system detection can be done automatically using a visualization system such as a camera or other detection system.

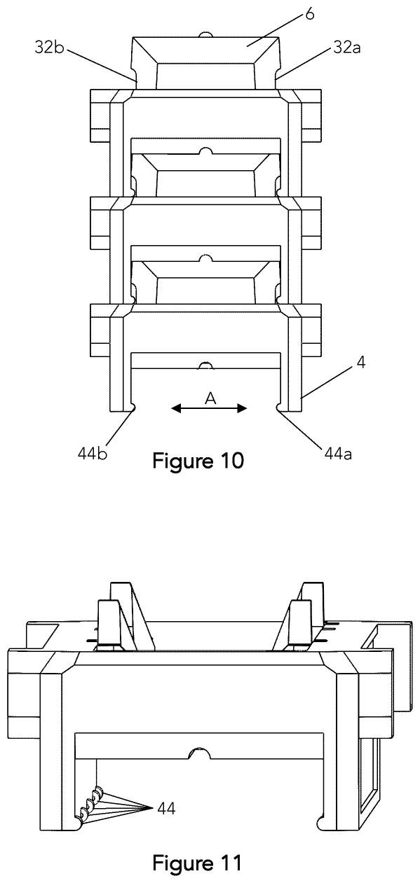

FIG. 10 is a front view of a stack of three flow assay cartridges. As shown, rail guides 44a, 44b fit into rail tracks 32a, 32b, respectively, to form a secure and reversible sliding engagement such that two cartridges can slide relative to one another. Sliding engagement of multiple cartridges provides an organized and safe way of transporting sets of cartridges from manufacturer to use site while limiting movement of cartridges to limit damage to the assay membrane strip housed inside. In addition, stacks of cartridges with related assay membrane assays can be packaged together for easy loading into an analyzer.

Each cartridge can also preferably be able to be friction snapped onto another cartridge by applying pressure to move rail guides 44a, 44b in the cartridge base 4 away from each other as shown by arrow ‘A’ such that they can fit over the wide top of cartridge lid 6 and be positioned into rail tracks 32a, 32b. This snap fit feature can be useful when cartridges in two stacks are desired to be fit together to be analysed in a single assay run by an analyzer, or when a single cartridge is desired to be added to an existing stack.

In use, cartridges as described can be provided with a wide variety of assay membranes having a wide variety of immobilized species in the detection area of the assay membrane. For example, different assay membranes can be placed into the cartridges for testing for different analytes of interest in a sample, allowing multiple tests to be done on a single sample in a single analyzer run. A variety of sets of assay cartridges can be available to point-of-care centres for testing, providing a lot of information about the contents of a particular sample by testing multiple analytes of interest at a time with a single cartridge set. For an environmental water sample, for example, the water sample can be tested for the presence of multiple microorganisms by providing a set of assay membranes each with a different immobilized species to detect one or more microorganisms. Applications of modular sets of vertical stacks of assay membrane cartridges can also be very useful in antibody testing for various antibodies using an automated analyzer. For example, in allergy testing, various sets of assay cartridges can be provided which are pre-prepared with a variety of antigens to test if a patient has the antibodies for a particular antigen. A set of cartridges for “pet” allergies can be provided to a point of care centre that has individual cartridges to test for the presence of, for example, cat dander, dog dander, horse dander, rodent dander. In another set of assay cartridges for “food” allergies, the set can comprise individual cartridges for testing the allergens that cause the majority of food allergies, specifically milk, eggs, peanuts, tree nuts, soy, wheat, fish, and shellfish. For a patient who is suspected of having both food and pet allergies, the two sets of cartridges can be provided to the point-of-care center and engageably stacked together such that the allergy tests for all of the allergens in both sets can be done in a single analyzer run with a single biological sample from the patient. Other allergy kits with different sets of allergens can also be provided, such as, for example, for drug allergy, insect allergy, latex allergy, grass allergy, mold allergy, metal allergy, and pollen allergy, to name a few.

FIG. 11 is a front view of a cartridge base with engagement features. A plurality of rail guides 44 are shown which serve as bottom engagement features configured to slidingly engage with a rail track of another cartridge below it.

All publications, patents and patent applications mentioned in this specification are indicative of the level of skill of those skilled in the art to which this invention pertains and are herein incorporated by reference. The invention being thus described, it will be obvious that the same may be varied in many ways. Such variations are not to be regarded as a departure from the scope of the invention, and all such modifications as would be obvious to one skilled in the art are intended to be included within the scope of the following claims.

1. A flow assay cartridge comprising:
a cartridge base;
a cartridge lid engageable with the cartridge base;
a bottom engagement feature on the cartridge base; and
a top engagement feature,
wherein the bottom engagement feature of a first flow assay cartridge can be engaged with the top engagement feature of a second flow assay cartridge positioned below the first flow assay cartridge for releasable sliding engagement of the second flow assay cartridge to the first flow assay cartridge, the top engagement feature and bottom engagement feature comprising a rail track and at least one complementary rail guide.

2. The cartridge of claim 1, wherein the bottom engagement feature of the first flow assay cartridge is engageable with the top engagement feature of the second flow assay cartridge with a snap engagement.

3. (canceled)

4. The cartridge of claim 1, wherein the top engagement feature comprises at least one rail track and the bottom engagement feature comprises at least one rail guide.

5. (canceled)

6. The cartridge of claim 1, wherein the flow assay cartridge is vertically stackable with a plurality of similar cartridges.

7. The cartridge of claim 1, wherein when the first flow assay cartridge is vertically stacked with the second flow assay cartridge, the cartridge lid of the second flow assay cartridge is covered by the cartridge base of the first flow assay cartridge.

8. The cartridge of claim 1, further comprising a flow assay membrane in the flow assay cartridge.

9. The cartridge of claim 1, wherein the cartridge further comprises features for engagement with an analyzer.

10. The cartridge of claim 1, wherein the cartridge lid is reversibly engageable to the cartridge base.

11. The cartridge of claim 1, wherein the cartridge lid has a plurality of apertures.

12. A method of flow assay automation comprising:
slidingly disengaging a first assay cartridge from a second assay cartridge in a vertically engaged stack of assay cartridges;
applying sample into the first assay cartridge to begin the assay; and
reengaging the first assay cartridge to another assay cartridge in the plurality of vertically engaged stack of assay cartridges by slidingly engaging the first assay cartridge to an engagement feature in an assay cartridge in the vertically engaged stack of assay cartridges, the engagement feature comprising a rail or a rail track.

13. The method of claim 12, wherein disengaging the first assay cartridge from the vertically engaged stack of assay cartridges is done by an automated device.

14. (canceled)

15. The method of claim 12, further comprising analysing the results of the assay.

16. A diagnostic test device comprising:
a flow assay membrane; and
a flow assay cartridge for receiving the flow assay membrane, the cartridge comprising:
a cartridge base;
a cartridge lid engageable with the cartridge base;
a bottom engagement feature on the cartridge base; and
a top engagement feature,
wherein the bottom engagement feature of a first flow assay cartridge can be engaged with the top engagement feature of a second flow assay cartridge positioned below the first flow assay cartridge for releasable sliding engagement of the second flow assay cartridge to the first flow assay cartridge, the bottom engagement feature and the top engagement feature comprising at least one rail track and at least one rail guide.

17. The test device of claim 16, further comprising a mounting locus where an analyzer component can engage with the assay cartridge for secure transport in the analyzer.

18-19. (canceled)

20. The test device of claim 16, wherein the bottom engagement feature and the top engagement feature comprise at least two rail tracks and at least two rail guides.
